(19) United States
(12) Patent Application Publication
(10) Pub. No.: US 2026/0001829 A1
(43) Pub. Date: Jan. 1, 2026

(54) METHOD FOR PRODUCING ACETONE

(71) Applicant: NIPPON SHOKUBAI CO., LTD., Osaka-shi, Osaka (JP)

(72) Inventor: Nao IGATA, Himeji-shi, Hyogo (JP)

(21) Appl. No.: 18/993,212

(22) PCT Filed: Aug. 3, 2023
(86) PCT No.: PCT/JP2023/028473

§ 371 Date: Jan. 10, 2025

(30) Foreign Application Priority Data
Aug. 3, 2022 (JP) 2022-124186

(51) Int. Cl. C07C 45/38

(52) U.S. Cl. CPC C07C 45/38

(57) ABSTRACT

The objective of the present invention is to provide a method for stably producing acetone using ethanol and water as starting materials while maintaining a high space time yield. The method for producing acetone comprises a step of synthesizing acetone by contacting ethanol with water in the presence of a catalyst, wherein the step of synthesizing acetone uses a reactant gas containing ethanol, water, and oxygen as a starting material, with the reactant gas having an oxygen concentration of 0.1 mol % to 10 mol %.

### TECHNICAL FIELD

The present invention relates to a method for producing acetone from ethanol, water, and oxygen.

### BACKGROUND ART

There have been several reports on the synthesis of acetone from ethanol and water. For example, Patent Literature 1 studies synthesis of acetone in the presence of a catalyst made of iron and zirconium and at a reaction temperature of 400° C. or higher. Further, Patent Literature 2 reports a method for producing acetone from ethanol and water in the presence of a catalyst containing iron, zinc, and an alkali metal and/or an alkaline earth metal, wherein the molar ratio of alkali metal and/or alkaline earth metal to zinc is 0.2 to 2.

### CITATION LIST

### Patent Literature

Patent Literature 1: JP 2009-209059 A

Patent Literature 2: JP 2012-240913 A

### SUMMARY OF INVENTION

### Technical Problem

Several methods for producing acetone from ethanol and water, including the above-mentioned methods, have been known. However, none of the methods are capable of stably producing acetone while maintaining a high space time yield, and there is room for improvement in these methods.

The present invention has been made in consideration of the above problems, and aims to provide a method for producing acetone from ethanol and water, which can stably produce acetone while maintaining a high space time yield.

### Solution to Problem

As a result of intensive studies to solve the above problems, the present inventor has found that use of a reactant gas containing oxygen at a specified concentration in the production of acetone from ethanol and water enables stable production of acetone while achieving a high space time yield condition.

Specifically, the present invention is as follows.
(1) A method for producing acetone, comprising a step of synthesizing acetone by contacting ethanol with water in the presence of a catalyst, the step of synthesizing acetone including using a reactant gas containing ethanol, water, and oxygen as a starting material, with the reactant gas having an oxygen concentration of 0.1 mol % to 10 mol %.
(2) The method for producing acetone according to (1), wherein a molar ratio of water to ethanol is 0.50 or higher and 10 or lower.
(3) The method for producing acetone according to (1) or (2), wherein a molar ratio of oxygen to ethanol is 0.01 or higher and lower than 1.4.
(4) The method for producing acetone according to any one of (1) to (3), wherein a total vaporization energy of the water and the ethanol per mole of the ethanol is 700 kW or less.
(5) The method for producing acetone according to any one of (1) to (4), wherein the catalyst contains at least one of a rare earth element or a transition metal element other than rare earth elements.
(6) The method for producing acetone according to any one of (1) to (5), wherein the ethanol and the water are contacted at a temperature of 250° C. to 600° C.
(7) The method for producing acetone according to any one of (1) to (6), wherein a space velocity of the reactant gas is 100 h⁻¹ to 10,000 h⁻¹.
(8) The method for producing acetone according to any one of (1) to (7), wherein the ethanol includes biomass-derived ethanol.

### Advantageous Effects of Invention

The method for producing acetone of the present disclosure can stably produce acetone using ethanol and water as starting materials while maintaining a high space time yield.

### DESCRIPTION OF EMBODIMENTS

The present disclosure is described in detail below.

Combinations of two or more of the individual preferred embodiments of the present disclosure described below are also preferred embodiments of the present disclosure. Herein, the phrase “X to Y” indicating a range means “X or more and Y or less.”

### Method for Producing Acetone of the Present Disclosure

### Catalyst

In the production method of the present disclosure, any catalyst capable of producing acetone from ethanol and water may be used. Preferably, the catalyst achieves a high acetone yield.

Non-limiting examples of the catalyst disclosed herein include a metal oxide containing a metal element, a support containing a metal element, and a support carrying a simple substance of a metal element or a metal oxide.

The metal oxide may be an oxide of one kind of metal element or a complex oxide containing two or more kinds of metal elements. Examples of the complex metal oxide include oxides having a crystalline structure such as spinel-type, perovskite-type, magnetoplumbite-type, or garnet-type, oxides having an amorphous structure, and oxides having both a crystalline portion and an amorphous portion.

Examples of the support include activated carbon, silica, alumina, silica-alumina, zeolite, silica-calcia, zirconia, ceria, magnesia, and diatomaceous earth. The catalyst herein refers to a catalyst in a state before the start of the reaction.

A transition metal element other than rare earth element and/or a rare earth element are preferably included as the metal element contained in the catalyst disclosed herein.

The transition metal element other than rare earth metal elements in the catalyst disclosed herein is preferably a metal element of Group 3, 4, 8, 11, or 12 of the periodic table, with zirconium, iron, copper, or zinc being more preferred.

The rare earth metal element in the catalyst disclosed herein is preferably lanthanum, praseodymium, or neodymium, with lanthanum being more preferred.

The catalyst disclosed herein preferably contains copper and/or iron, zirconium, and further contains a transition metal element other than copper, iron, and zirconium excluding rare earth elements and/or a rare earth element. More preferably, the catalyst contains copper and/or iron and zirconium, and further contains zinc or lanthanum.

The content of the metal element contained in the catalyst disclosed herein is preferably 0.1% by mass to 95% by mass, more preferably 30% by mass to 90% by mass, still more preferably 50% by mass to 87% by mass, and particularly preferably 60% by mass to 87% by mass, relative to the total amount of the catalyst. In a preferred form of the catalyst disclosed herein, the content of the metal element contained in the catalyst is 1 to 75% by mass.

The content of the metal element contained in the catalyst disclosed herein refers to the content of the metal element contained in the catalyst before the start of the reaction.

The content of the metal element can be measured by X-ray fluorescence analysis (XRF). Specifically, it may be measured by the method described in JIS K 0119:2008.

The shape of the catalyst and support used in the method for producing acetone of the present disclosure may be any shape. Examples of the shape include a spherical shape, a pellet shape, a honeycomb shape, a ring shape, and a granular shape.

The size of the catalyst used in the method for producing acetone of the present disclosure may be any size. The average particle size of the catalyst is preferably 1 mm to 12 mm, more preferably 3 mm to 10 mm. When the average particle diameter of the catalyst is within the above range, the catalyst can be easily packed into a reaction tube, and the pressure loss in the catalyst layer can be reduced. Thereby, energy savings such as a reduction in the power cost of the blower can be achieved. The average particle size of the catalyst can be determined by measuring the particle sizes of 100 randomly sampled particles of the catalyst with a vernier caliper and calculating the average value. Here, the particle size of the catalyst refers to, in the case of a spherical catalyst, the diameter of the catalyst, and refers to, in the case of a catalyst with another shape, the diameter of the circumscribed sphere of the catalyst.

### Contact Between Ethanol, Water, and Oxygen

The method for producing acetone of the present disclosure includes a step of synthesizing acetone by contacting ethanol with water in the presence of a catalyst (hereinafter, the contact may also be referred to as a reaction) (hereinafter, the step of synthesizing acetone may also be referred to as a reaction step).

In the method for producing acetone of the present disclosure, oxygen (hereinafter sometimes referred to as molecular oxygen), ethanol, and water are used as starting materials in the reaction step, and the ethanol and water are contacted in the presence of a catalyst in an oxygen-containing atmosphere. Thereby, a reaction product containing acetone, hydrogen, and carbon dioxide can be obtained.

The method for producing acetone of the present disclosure is not limited and may be either a batch method or a continuous method, with a continuous method being preferred from the viewpoint of productivity.

The method for producing acetone of the present disclosure preferably involves a gas phase reaction. Examples of a reaction system of the gas phase reaction include a fixed-bed system, a moving-bed system, and a fluidized-bed system. A fixed-bed system, which is simpler, is preferred.

When the method for producing acetone of the present disclosure is carried out in a fixed bed system, a premix of ethanol, water (sometimes referred to as steam), and oxygen may be supplied to the reactor as a reaction gas (hereinafter sometimes referred to as starting material gas) before contacting with the catalyst and contacted with the catalyst. Alternatively, any two of ethanol, water, and oxygen may be premixed, and the remaining one may be supplied separately to the reactor. Alternatively, each of the three, gaseous ethanol, steam, and molecular oxygen, may be supplied separately to the reactor. Preferably, any two of ethanol, water, and oxygen are premixed and the remaining one is separately supplied to the reactor, and more preferably, ethanol and water are premixed and oxygen is separately supplied to the reactor. Here, the reactant gas usually refers to the gas at the inlet of the reactor.

When the method for producing acetone of the present disclosure involves a gas-phase catalytic reaction, it may be a common single flow method or a recycle method.

The ethanol disclosed herein may be gaseous or misty, and is preferably gaseous. Gaseous ethanol can be obtained, for example, by heating liquid ethanol in a vaporizer.

The water disclosed herein may be gaseous or misty, and is preferably gaseous. Gaseous water can be obtained, for example, by heating water in a vaporizer.

The starting material gas contains oxygen (molecular oxygen), and may contain an inert gas such as nitrogen or helium in addition to oxygen. Here, the starting material gas includes all gases to be fed to the reactor.

The concentration of ethanol in the starting material gas is preferably 3 mol % or higher, more preferably 5 mol % or higher, particularly preferably 8 mol % or higher. When the concentration is 3 mol % or higher, acetone can be produced efficiently.

The concentration of ethanol in the starting material gas is preferably 66 mol % or lower, more preferably 50 mol % or lower, still more preferably 30 mol % or lower, particularly preferably 10 mol % or lower. When the concentration is 66 mol % or lower, a sufficient amount of oxygen can be allowed to coexist with ethanol.

In other words, the concentration of ethanol in the starting material gas is preferably 3 to 66 mol %, more preferably 3 to 50 mol %, still more preferably 5 to 30 mol %, particularly preferably 8 to 10 mol %.

The concentration of water in the starting material gas is preferably 20 mol % or higher, more preferably 30 mol % or higher, still more preferably 33 mol % or higher, particularly preferably 35 mol % or higher. When the concentration is 20 mol % or higher, acetone can be produced efficiently.

The concentration of water in the starting material gas is preferably 80 mol % or lower, more preferably 59 mol % or lower, still more preferably 50 mol % or lower, particularly preferably 44 mol % or lower. When the concentration is 80 mol % or lower, the total vaporization energy of the water and the ethanol per mole of the ethanol can be kept small, and acetone can be produced at low cost.

In other words, the concentration of water in the starting material gas is preferably 20 mol % to 80 mol %, more preferably 30 mol % to 80 mol %, still more preferably 33 mol % to 59 mol %, particularly preferably 35 mol % to 50 mol %, most preferably 35 mol % to 44 mol %. When the concentration of water in the starting material gas is within these ranges, a higher acetone yield can be achieved.

In the starting material gas used in the method for producing acetone of the present disclosure, the molar ratio of water to ethanol is preferably 0.5 or higher, more preferably 2.5 or higher, still more preferably 3.5 or higher. The molar ratio of water to ethanol of 0.5 or higher is preferred because a high acetone selectivity is achieved.

In addition, in the starting material gas used in the method for producing acetone of the present disclosure, the molar ratio of water to ethanol is preferably 10 or lower, more preferably 7.0 or lower, still more preferably 4.5 or lower. The molar ratio of oxygen to ethanol of 10 or lower is preferred because the total vaporization energy of the water and the ethanol per mole of the ethanol can be kept small, and acetone can be produced at low cost.

In other words, in the starting material gas used in the method for producing acetone of the present disclosure, the molar ratio of water to ethanol is preferably 0.5 to 10, more preferably 0.5 to 7.0, still more preferably 2.5 to 7.0, particularly preferably 3.5 to 4.5.

In the starting material gas used in the method for producing acetone of the present disclosure, the total vaporization energy of the water and the ethanol per mole of the ethanol is preferably 700 kW or less. More preferably, the total vaporization energy is 350 kW or less, still more preferably 150 kW or less. The lower the total vaporization energy of the water and the ethanol per mole of the ethanol, the lower the cost of synthesizing acetone. Yet, since the reaction proceeds efficiently by vaporizing water, the total vaporization energy of the water and the ethanol per mole of the ethanol is preferably more than 0.

Commonly, the total vaporization energy of the water and the ethanol per mole of the ethanol is 75 kW or more.

Any ethanol may be used in the starting material gas. Examples thereof include ethanol obtained by ethylene hydration and bioethanol made from biomass feedstocks such as carbohydrates (e.g., sugar cane), starches (e.g., grains), and celluloses (e.g., plants).

The ethanol used in the starting material gas preferably includes bioethanol. The amount of bioethanol in 100% by mass of the ethanol used in the starting material gas (also referred to as bioethanol content) is preferably 50% by mass or more, more preferably 75% by mass by mass or more, still more preferably 90% by mass or more.

The bioethanol content can be measured as follows.
1. The ethanol used in the starting material gas is burned, converting the whole amount of the ethanol into carbon dioxide.
2. The carbon dioxide is separated and purified using a vacuum line.
3. The whole amount of the carbon dioxide produced from the ethanol is reduced with hydrogen in the presence of iron as a catalyst to produce graphite.
4. Using a ¹⁴C-AMS measurement system available from NEC, the ratio of ¹⁴C concentration to ¹³C concentration (¹⁴C/¹³C) of the ethanol-derived graphite is measured.
5. Oxalic acid (hereinafter also referred to as a reference sample) produced in the same year as the raw ethanol provided by the National Institute of Standards and Technology (NIST) is subjected to measurement of the ratio of ¹⁴C concentration to ¹³C concentration (¹⁴C/¹³C) by the same procedures as 1 to 4 above.
6. The ¹⁴C/¹³C value of the graphite derived from the raw ethanol is divided by the ¹⁴C/¹³C value of the reference sample, and the resulting value is multiplied by 100 to obtain the bioethanol content.

The concentration of molecular oxygen in the starting material gas is preferably 0.1 mol % or higher, more preferably 0.5 mol % or higher, still more preferably 0.7 mol % or higher, particularly preferably 1 mol % or higher. The concentration of 0.1 mol % or higher is preferred because a decrease in the temperature of the catalyst layer due to the endothermic reaction between ethanol and water can be prevented or reduced, thereby improving the acetone yield.

The concentration of molecular oxygen in the starting material gas is preferably 10 mol % or lower, more preferably 7.5 mol % or lower, still more preferably 7 mol % or lower, particularly preferably 5 mol % or lower. The concentration of 10 mol % or lower is preferred because a decrease in the acetone yield due to combustion of the produced acetone and ethanol by oxygen can be prevented or reduced and acetone can be obtained in a high yield by selectively combusting the produced hydrogen.

The concentration of molecular oxygen in the starting material gas is preferably 0.1 mol % to 10 mol %, more preferably 0.5 mol % to 7.5 mol, still more preferably 0.7 mol % to 7 mol %, particularly preferably 1 mol % to 5 mol %. The concentration within the above ranges is preferred because combustion of the ethanol and the acetone with excess oxygen is prevented or reduced and the temperature distribution in the catalyst layer is reduced, thereby improving the acetone yield.

In the starting material gas used in the method for producing acetone of the present disclosure, the molar ratio of oxygen to ethanol is preferably 0.01 or higher, more preferably 0.05 or higher, still more preferably 0.1 or higher. The molar ratio of oxygen to ethanol of 0.01 or higher is preferred because a decrease in the temperature of the catalyst layer is prevented or reduced and the acetone yield is improved.

In addition, in the starting material gas used in the method for producing acetone of the present disclosure, the molar ratio of oxygen to ethanol is preferably 1.4 or lower, more preferably 0.8 or lower, still more preferably 0.3 or lower. The molar ratio of oxygen to ethanol of 1.4 or lower is preferred because the combustion of the ethanol and the acetone can be prevented or reduced and the acetone yield does not decrease.

In other words, in the starting material gas used in the method for producing acetone of the present disclosure, the molar ratio of oxygen to ethanol is preferably 0.01 to 1.4, more preferably 0.05 to 0.8, still more preferably 0.1 to 0.3.

In the reaction step of the method for producing acetone of the present disclosure, the reaction pressure may be reduced pressure, atmospheric pressure, or increased pressure, and is preferably 0.07 MPa to 0.2 MPa, more preferably 0.1 MPa to 0.15 MPa.

In the reaction step of the production method of the present disclosure, the temperature at which the ethanol, water, and oxygen are contacted, i.e., the temperature of the reaction between the ethanol and the water, is preferably 250° C. to 600° C., more preferably 300° C. to 550° C., still more preferably 330° C. to 500° C., further more preferably 350° C. to 450° C., particularly preferably 365° C. to 435° C., most preferably 365° C. to 415° C. When the reaction is performed at such temperatures, a decrease in catalytic activity over time tends to be prevented or reduced.

Since in the method for producing acetone of the present disclosure, the reaction is performed using a catalyst, the temperature of the reaction between the ethanol and the water herein refers to the average temperature of the catalyst layer. The average temperature of the catalyst layer is an average of the temperatures measured at 10 or more points at equal intervals in the gas flow direction from the inlet to the outlet of the catalyst layer.

As for the temperature difference inside the catalyst layer in the method for producing acetone of the present disclosure, the temperature difference between a site with the highest temperature and a site with the lowest temperature inside the catalyst layer is preferably 100° C. or less, more preferably 70° C. or less, still more preferably 50° C. or less.

In the method for producing acetone of the present disclosure, the space velocity of the reactant gas is preferably 100 h⁻¹ to 10,000 h⁻¹, more preferably 300 h⁻¹ to 9, 000 h⁻¹, still more preferably 500 h⁻¹ to 8,000 h⁻¹, particularly preferably 900 h⁻¹ to 6, 000 h⁻¹, most preferably 2,500 h⁻¹ to 5,000 h⁻¹. Commonly, the higher the space velocity of the reactant gas, the more difficult it is for the reaction to proceed sufficiently. The method for producing acetone of the present disclosure allows the reaction of synthesizing acetone from ethanol to proceed sufficiently even when the space velocity of the reactant gas is high. Thus, use of a reactant gas with such a space velocity in the reaction enables production of a larger amount of acetone per unit time.

In the method for producing acetone of the present disclosure, the space time yield is preferably 300 kg/(m³·h) or more, more preferably 575 kg/(m³·h) or more, still more preferably 775 kg/(m³·h) or more.

The reason why acetone can be stably produced while maintaining a high space time yield in the present invention is mainly presumed to be due to the following (1) and (2).
(1) When producing acetone from ethanol and water, the by-product hydrogen reacts with oxygen in the reactant gas to form water, which generates heat. This offsets heat absorbed in the production of acetone from ethanol and water. (This contributes to the stable progress of the reaction of producing acetone from ethanol and water.)
(2) The reactant gas contains a moderate concentration of molecular oxygen, so that most of the molecular oxygen is consumed by combustion of hydrogen. (Since most of the molecular oxygen is not used for combustion of ethanol or acetone, most of the ethanol in the reactant gas is reacted with water. In addition, most of the acetone produced can be collected as a product.)

It goes without saying that these mechanisms are merely speculation and do not limit the technical scope of the present invention.

### Other Steps

The method for producing acetone of the present disclosure may include steps other than the reaction step. Examples of the other steps include a purification step, and a catalyst regeneration step, etc.

The method for producing acetone of the present disclosure preferably achieves a higher ethanol conversion, a higher acetone selectivity, and a higher acetone yield. The ethanol conversion is preferably 89% or higher, and the acetone selectivity and acetone yield are each preferably 50% or higher.

The values of ethanol conversion, acetone selectivity, and acetone yield can be determined by the methods described in the Examples below.

### Acetone Production Apparatus in the Present Disclosure

The method for producing acetone of the present disclosure is performed using a production apparatus, preferably a fixed-bed reactor. The production apparatus may be a fixed-bed reactor connected to a vaporizer for obtaining a starting material gas.

The production apparatus may be made of any material, and is preferably made of stainless steel. Typical examples of stainless steel include austenitic stainless steels such as SUS304, SUS304L, SUS316, and SUS316L according to the Japanese Industrial Standards (hereinafter also referred to as JIS).

### Applications of Acetone in the Present Disclosure

Acetone produced by the method for producing acetone of the present disclosure may be used for any application, and is suitably used for a starting material for producing isopropyl alcohol. The acetone produced by the method for producing acetone of the present disclosure may be hydrogenated by a known method, for example, to produce isopropyl alcohol. The present invention also encompasses a method for producing isopropyl alcohol, including producing acetone by the method for producing acetone of the present disclosure and hydrogenating the acetone to produce isopropyl alcohol.

### EXAMPLES

The present invention is described in more detail below with reference to examples. However, the present invention is not limited to the following examples, and it is of course possible to carry out the present invention with appropriate modifications within the scope of the above and below-mentioned spirit, and all of these modifications are included in the technical scope of the present invention.

### Synthesis Example 1

To 3800 g of pure water were added 629 g of zinc nitrate hexahydrate, 562 g of zirconium oxynitrate dihydrate, 1700 g of iron nitrate nonahydrate, 32.8 g of cesium nitrate, and 1545 g of 28% by weight aqueous ammonia, followed by stirring for 20 hours to obtain a starting material liquid mixture. The starting material liquid mixture was dried in a drum dryer at 150° C., and the dried product was then pulverized and sieved to 150 um or less to obtain a catalyst precursor powder. The catalyst precursor powder was calcined at 450° C. for four hours in an air atmosphere to obtain a catalyst powder. Then, 500 g of the catalyst powder, 5 g of hydroxyethyl cellulose, and 100 g of water were placed in an extruder and molded into a cylindrical shape of 6 mm in diameter and 6 mm in length to obtain a catalyst molding to be calcined. The catalyst molding to be calcined was calcined at 450° C. for four hours in an air atmosphere to obtain a catalyst (FeZnZro catalyst: Fe₂ZnZrO₆).

### Synthesis Example 2

To 1500 g of pure water were added 498 g of lanthanum nitrate hexahydrate, 308 g of zirconium oxynitrate dihydrate, 1000 g of copper nitrate trihydrate, and 926 g of 85% by weight potassium hydroxide, followed by stirring for 20 hours to obtain a starting material liquid mixture. The starting material liquid mixture was filtered, and the filtration residue was washed with pure water until the pH of the filtrate fell within the range of 6 to 8. The washed filtration residue was placed in a dryer at 120° C. and dried for 20 hours. The resulting dried product was pulverized and sieved to 150 μm or less to obtain a catalyst precursor powder. The catalyst precursor powder was calcined at 450° C. for four hours in an air atmosphere to obtain a catalyst powder. Then, 500 g of the catalyst powder, 5 g of hydroxyethyl cellulose, and 100 g of water were placed in an extruder and molded into a cylindrical shape of 6 mm in diameter and 6 mm in length to obtain a catalyst molding to be calcined. The catalyst molding to be calcined was calcined at 450° C. for four hours in an air atmosphere to obtain a catalyst (CuLaZro catalyst: Cu₂La₂ZrO₇).

### Example 1

Acetone was produced using the catalyst synthesized in Synthesis Example 1 using a U-shaped reactor made of SUS316 (outer diameter 25.6 mm, inner diameter 21.6 mm). The U-shaped reactor equipped with a stainless steel thermometer protective tube with an outer diameter of 3 mm in the center of the reactor was filled with 140 g of the catalyst. The packed catalyst layer had a length of 340 mm. The reaction tube filled with the catalyst was placed in a molten salt bath, nitrogen was fed at 5.2 L/min (in terms of 0° C. and 1 atm), and the temperature of the molten salt bath was raised to 375° C. and maintained for 30 minutes. Thereafter, nitrogen, ethanol, and water (water vapor) were fed as reactant gases at 5.0 L/min (in terms of 0° C. and 1 atm), 1.0 L/min (in terms of 0° C. and 1 atm), and 4.0 L/min (in terms of 0° C. and 1 atm), respectively, for reaction. One hour after the start of the feeding of the reactant gases, while the flow rates of the ethanol and the water were maintained at 1.0 L/min (in terms of 0° C. and 1 atm) and 4.0 L/min (in terms of 0° C. and 1 atm), respectively, the flow rate of the nitrogen was reduced to 4.0 L/min (in terms of 0° C. and 1 atm), and air was fed at 1.0 L/min (in terms of 0° C. and 1 atm). The ethanol conversion, acetone selectivity, and acetone yield two hours after the start of the reaction were measured. In addition, the total vaporization energy of the water and the ethanol per mole of the ethanol was calculated.

Here, the ethanol conversion, acetone selectivity, and acetone yield were calculated according to the formulas (1), (2), and (3).

$$\begin{matrix}[ Math . 1 ] & \\ Ethanol conversion = 100 - 100 \times Ethanol flow rate at reactor outlet / Ethanol flow rate at reactor inlet & ( 1 )\end{matrix}$$

$$\begin{matrix}[ Math . 2 ] & \\ Acetone selectivity = ( Acetone flow rate at reactor outlet \times 3 / ( Ethanol flow rate at reactor inlet \times 2 ) ) / ( Ethanol conversion ) & ( 2 )\end{matrix}$$

$$\begin{matrix}[ Math . 3 ] & \\ Acetone yield = 100 \times Aceton flow rate reactor outlet \times 3 / ( Ethanol flow rate at reactor inlet \times 2 ) & ( 3 )\end{matrix}$$

The synthesis reaction of acetone from ethanol and water is represented by the following reaction formula (4).

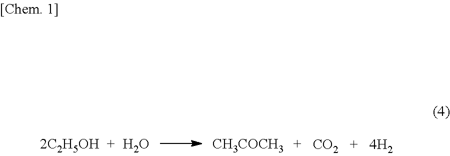

The acetone yield represented by the formula (3) is evaluated by the amount of carbon in the produced acetone relative to the total amount of carbon in the ethanol fed to the reactor inlet. Thus, the maximum acetone yield was 75%.

The gas at the reactor outlet was introduced into an absorption bottle containing pure water placed in an ice-water bath, and the components captured by the water were quantitatively determined with a gas chromatograph. The gas at the outlet of the absorption bottle was introduced into a gas chromatograph, whereby components not collected in the pure water in the absorption bottle were quantified. From the resulting analytical values, the flow rates of the components in the gas at the reactor outlet were calculated, and the ethanol conversion, acetone selectivity, and acetone yield were calculated using the formulas (1), (2), and (3). The temperature of the catalyst layer was determined by measuring the temperatures at 34 points at intervals of 10 mm from the inlet to the outlet of the catalyst layer and obtaining the average of these temperatures to determine an average temperature (° C.) of the catalyst layer.

The vaporization energies of the water and the ethanol per mole of the ethanol were calculated from the flow rates of the corresponding gases at the reactor inlet and the reaction temperatures using the chemical process simulator COCO/ChemSep.

### Example 2

A reaction was performed as in Example 1, except that the ethanol was changed to biomass-derived ethanol, and that one hour after the start of the feeding of the reactant gases, the flow rates of the nitrogen, ethanol, and water were set to 0 L/min (in terms of 0° C. and 1 atm), 1.0 L/min (in terms of 0° C. and 1 atm), and 4.0 L/min (in terms of 0° C. and 1 atm), respectively, and that air was fed at 0.05 L/min (in terms of 0° C. and 1 atm). The ethanol conversion, acetone selectivity, and acetone yield two hours after the start of the reaction were measured.

### Example 3

A reaction was performed as in Example 1, except that the ethanol was changed to biomass-derived ethanol. The ethanol conversion, acetone selectivity, and acetone yield two hours after the start of the reaction were measured.

### Example 4

A reaction was performed as in Example 1, except that the ethanol was changed to biomass-derived ethanol, and that one hour after the start of the feeding of the reactant gases, the flow rates of the nitrogen, ethanol, and water were set to 2.5 L/min (in terms of 0° C. and 1 atm), 1.0 L/min (in terms of 0° C. and 1 atm), and 4.0 L/min (in terms of 0° C. and 1 atm), respectively, and that air was fed at 2.5 L/min (in terms of 0° C. and 1 atm). The ethanol conversion, acetone selectivity, and acetone yield two hours after the start of the reaction were measured.

### Example 5

A reaction was performed as in Example 1, except that the ethanol was changed to biomass-derived ethanol, and that one hour after the start of the feeding of the reactant gases, the flow rates of the nitrogen, ethanol, and water were set to 0 L/min (in terms of 0° C. and 1 atm), 1.0 L/min (in terms of 0° C. and 1 atm), and 1.0 L/min (in terms of 0° C. and 1 atm), respectively, and that air was fed at 0.23 L/min (in terms of 0° C. and 1 atm). The ethanol conversion, acetone selectivity, and acetone yield two hours after the start of the reaction were measured.

### Example 6

A reaction was performed as in Example 1, except that the ethanol was changed to biomass-derived ethanol, and that one hour after the start of the feeding of the reactant gases, the flow rates of the nitrogen, ethanol, and water were set to 0 L/min (in terms of 0° C. and 1 atm), 1.0 L/min (in terms of 0° C. and 1 atm), and 1.0 L/min (in terms of 0° C. and 1 atm), respectively, and that air was fed at 2.0 L/min (in terms of 0° C. and 1 atm). The ethanol conversion, acetone selectivity, and acetone yield two hours after the start of the reaction were measured.

### Example 7

A reaction was performed as in Example 1, except that the ethanol was changed to biomass-derived ethanol, and that one hour after the start of the feeding of the reactant gases, the flow rates of the nitrogen, ethanol, and water were set to 0.1 L/min (in terms of 0° C. and 1 atm), 1.0 L/min (in terms of 0° C. and 1 atm), and 0.5 L/min (in terms of 0° C. and 1 atm), respectively, and that air was fed at 0.2 L/min (in terms of 0° C. and 1 atm). The ethanol conversion, acetone selectivity, and acetone yield two hours after the start of the reaction were measured.

### Example 8

A reaction was performed as in Example 1, except that the ethanol was changed to biomass-derived ethanol, and that one hour after the start of the feeding of the reactant gases, the flow rates of the nitrogen, ethanol, and water were set to 5.0 L/min (in terms of 0° C. and 1 atm), 1.0 L/min (in terms of 0° C. and 1 atm), and 12.0 L/min (in terms of 0° C. and 1 atm), respectively, and that air was fed at 2.0 L/min (in terms of 0° C. and 1 atm). The ethanol conversion, acetone selectivity, and acetone yield two hours after the start of the reaction were measured. It was found that due to the large amount of water, the total vaporization energy of the water and the ethanol per mole of the ethanol was large, and acetone could not be produced at low cost. It was also found that if vaporization was insufficient, there was a risk of electric leakage in the vaporizer and other equipment due to water leakage. Therefore, the reaction was stopped.

### Example 9

A reaction was performed as in Example 1, except that the ethanol was changed to biomass-derived ethanol, and that one hour after the start of the feeding of the reactant gases, the flow rates of the nitrogen, ethanol, and water were set to 4.0 L/min (in terms of 0° C. and 1 atm), 1.0 L/min (in terms of 0° C. and 1 atm), and 4.0 L/min (in terms of 0° C. and 1 atm), respectively, and that air was fed at 1.0 L/min (in terms of 0° C. and 1 atm). The ethanol conversion, acetone selectivity, and acetone yield two hours after the start of the reaction were measured.

### Example 10

A reaction was performed as in Example 1, except that the catalyst was changed to the catalyst synthesized in

Synthesis Example 2, that the ethanol was changed to biomass-derived ethanol, and that one hour after the start of the feeding of the reactant gases, the flow rates of the nitrogen, ethanol, and water were set to 0 L/min (in terms of 0° C. and 1 atm), 1.0 L/min (in terms of 0° C. and 1 atm), and 4.0 L/min (in terms of 0° C. and 1 atm), respectively, and that air was fed at 0.05 L/min (in terms of 0° C. and 1 atm). The ethanol conversion, acetone selectivity, and acetone yield two hours after the start of the reaction were measured.

### Example 11

A reaction was performed as in Example 1, except that the catalyst was changed to the catalyst synthesized in Synthesis Example 2, that the ethanol was changed to biomass-derived ethanol, and that one hour after the start of the feeding of the reactant gases, the flow rates of the nitrogen, ethanol, and water were set to 3.8 L/min (in terms of 0° C. and 1 atm), 1.0 L/min (in terms of 0° C. and 1 atm), and 4.0 L/min (in terms of 0° C. and 1 atm), respectively, and that air was fed at 1.2 L/min (in terms of 0° C. and 1 atm). The ethanol conversion, acetone selectivity, and acetone yield two hours after the start of the reaction were measured.

### Comparative Example 1

The same stainless steel U-shaped reaction tube as that used in Example 1 was filled with 140 g of the catalyst synthesized in Synthesis Example 1. The reaction tube filled with the catalyst was placed in a molten salt bath, nitrogen was fed at 5.2 L/min (in terms of 0° C. and 1 atm), and the temperature of the molten salt bath was raised to 375° C. and maintained for 30 minutes. Thereafter, nitrogen, ethanol, and water (water vapor) were fed at 5.0 L/min (in terms of 0° C. and 1 atm), 1.0 L/min (in terms of 0° C. and 1 atm), and 4.0 L/min (in terms of 0° C. and 1 atm), respectively, for reaction. The ethanol conversion and acetone yield two hours after the start of the reaction were measured as in Example 1.

### Comparative Example 2

The ethanol was changed to biomass-derived ethanol, and one hour after the start of the feeding of the reactant gases, the flow rates of the nitrogen, ethanol, and water were set to 0 L/min (in terms of 0° C. and 1 atm), 1.0 L/min (in terms of 0° C. and 1 atm), and 4.0 L/min (in terms of 0° C. and 1 atm), respectively, and air was fed at 7.5 L/min (in terms of 0° C. and 1 atm). However, due to the mixing ratio of the gases in the gas mixture containing components such as ethanol and oxygen, there was a risk of explosion. Therefore, the reaction was stopped.

### Comparative Example 3

A reaction was performed as in Example 1, except that the ethanol was changed to biomass-derived ethanol, and that one hour after the start of the feeding of the reactant gases, the flow rates of the nitrogen, ethanol, and water were set to 0.23 L/min (in terms of 0° C. and 1 atm), 1.0 L/min (in terms of 0° C. and 1 atm), and 1.0 L/min (in terms of 0° C. and 1 atm), respectively, and that air was fed at 0 L/min (in terms of 0° C. and 1 atm). The ethanol conversion, acetone selectivity, and acetone yield two hours after the start of the reaction were measured.

TABLE 1

| | | Concentration of reactant gas, ratio, etc. | | | | | | |
|---|---|---|---|---|---|---|---|---|
| | | | | | | | | Total |
| | | | | | | | | vaporization |
| | | | | | | | | energy of EtOH |
| | | | | | | Water/Ethanol | Oxygen/Ethanol | and water |
| | | Ethanol | Water | Nitrogen | Oxygen | (molar | (molar | [kW/(mol- |
| | Catalyst | (mol %) | (mol %) | (mol %) | (mol %) | ratio) | ratio) | EtOH · s)] |
| Example 1 | FeZnZrO | 10 | 40 | 48 | 2 | 4.00 | 0.20 | 305 |
| Example 2 | FeZnZrO | 20 | 79 | 0.8 | 0.2 | 3.95 | 0.01 | 302 |
| Example 3 | FeZnZrO | 10 | 40 | 48 | 2 | 4.00 | 0.20 | 305 |
| Example 4 | FeZnZrO | 10 | 40 | 45 | 5 | 4.00 | 0.50 | 305 |
| Example 5 | FeZnZrO | 45 | 45 | 8 | 2 | 1.00 | 0.04 | 134 |
| Example 6 | FeZnZrO | 25 | 25 | 40 | 10 | 1.00 | 0.40 | 134 |
| Example 7 | FeZnZrO | 55 | 27 | 16 | 2 | 0.49 | 0.04 | 105 |
| Example 8 | FeZnZrO | 5 | 60 | 33 | 2 | 12.00 | 0.40 | 761 |
| Example 9 | FeZnZrO | 10 | 40 | 48 | 2 | 4.00 | 0.20 | 305 |
| Example 10 | CuLaZrO | 20 | 79 | 0.8 | 0.2 | 3.95 | 0.01 | 302 |
| Example 11 | CuLaZrO | 10 | 40 | 48 | 2 | 4.00 | 0.20 | 305 |
| Comparative | FeZnZrO | 10 | 40 | 50 | 0 | 4.00 | 0.00 | 305 |
| Example 1 | | | | | | | | |
| Comparative | FeZnZrO | 8 | 32 | 48 | 12 | 4.00 | 1.50 | 305 |
| Example 2 | | | | | | | | |
| Comparative | FeZnZrO | 45 | 45 | 10 | 0 | 1.00 | 0.00 | 134 |
| Example 3 | | | | | | | | |

| | | | Reaction result | | | | |
|---|---|---|---|---|---|---|---|
| | | | | | | | Space |
| | Space velocity | | Average | | | | time |
| | All | | temperature | Ethanol | Acetone | Acetone | yield |
| | gases | Ethanol | of catalyst | conversion | selectivity | yield | (kg/ |
| | (h⁻¹) | (h⁻¹) | layer (° C.) | (%) | (%) | (%) | (m³ · h) |
| Example 1 | 4816 | 482 | 374 | 99.9 | 68.3 | 68.2 | 863 |
| Example 2 | 2408 | 482 | 362 | 89.0 | 69.7 | 62.0 | 785 |
| Example 3 | 4816 | 482 | 374 | 99.9 | 68.0 | 67.9 | 860 |
| Example 4 | 4816 | 482 | 423 | 100.0 | 62.1 | 62.1 | 786 |
| Example 5 | 1070 | 482 | 376 | 100.0 | 52.6 | 52.6 | 666 |
| Example 6 | 1926 | 482 | 420 | 100.0 | 50.2 | 50.2 | 635 |
| Example 7 | 876 | 482 | 377 | 63.0 | 41.7 | 26.3 | 332 |
| Example 8 | 9632 | 482 | — | — | — | — | — |
| Example 9 | 4816 | 482 | 441 | 100.0 | 42.3 | 42.3 | 535 |
| Example 10 | 2408 | 482 | 318 | 97.0 | 62.2 | 60.3 | 763 |
| Example 11 | 4816 | 482 | 325 | 100.0 | 61.3 | 61.3 | 776 |
| Comparative | 4816 | 482 | 362 | 88.9 | 68.5 | 60.9 | 771 |
| Example 1 | | | | | | | |
| Comparative | 6020 | 482 | — | — | — | — | — |
| Example 2 | | | | | | | |
| Comparative | 1070 | 482 | 365 | 86.3 | 56.3 | 48.6 | 615 |
| Example 3 | | | | | | | |

Table 1 shows the molar percentages of reactant gases, the total vaporization energy of the water and the ethanol per mole of the ethanol, and the reaction results. As can be seen from Table 1, when the reactant gas contained molecular oxygen, the average temperature of the catalyst layer was high, the reaction proceeded with a high ethanol conversion, and a product was obtained with an excellent acetone selectivity. In other words, it clearly demonstrated that acetone was produced stably while maintaining a high space time yield. The molar percentages of the reactant gases in Table 1 are the molar percentages of the gases in the reactant gas after the start of the feeding of air for Examples 1 to 11 and Comparative Example 2.

1. A method for producing acetone, comprising a step of synthesizing acetone by contacting ethanol with water in the presence of a catalyst,
the step of synthesizing acetone including using a reactant gas containing ethanol, water, and oxygen as a starting material, with the reactant gas having an oxygen concentration of 0.1 mol % to 10 mol %.

2. The method for producing acetone according to claim 1,
wherein a molar ratio of water to ethanol is 0.50 or higher and 10 or lower.

3. The method for producing acetone according to claim 1,
wherein a molar ratio of oxygen to ethanol is 0.01 or higher and lower than 1.4.

4. The method for producing acetone according to claim 1,
wherein a total vaporization energy of the water and the ethanol per mole of the ethanol is 700 kW or less.

5. The method for producing acetone according to claim 1,
wherein the catalyst contains at least one of a rare earth element or a transition metal element other than rare earth elements.

6. The method for producing acetone according to claim 1,
wherein the temperature at which the ethanol and the water are contacted is 250° C. to 600° C.

7. The method for producing acetone according to claim 1,
wherein a space velocity of the reactant gas is 100 h⁻¹ to 10,000 h⁻¹.

8. The method for producing acetone according to claim 1,
wherein the ethanol includes biomass-derived ethanol.
